(19) United States
(12) Patent Application Publication
(10) Pub. No.: US 2005/0027266 A1
(43) Pub. Date: Feb. 3, 2005

(54) Enema body fluid collecting bag

(76) Inventor: Jim D. Howlett, Abilene, TX (US)

Correspondence Address: Jim D. Howlett, P.O. Box 6258, Abilene, TX, 79608, US

(21) Appl. No.: 10/887,763

(22) Filed: Jul. 8, 2004

Related U.S. Application Data
(60) Provisional application No. 60/490,606, filed on Jul. 29, 2003.

(51) Int. Cl. A61M 1/00

(52) U.S. Cl. 604/317

(57) ABSTRACT

The E.B.F. Bag will capture the body fluids/waste material throughout the complete process as the enema is administered, eliminating contamination of the patient, bed, linens, and floor as well as the caregiver and others that administer the enema. This bag can be administered to patients that are in a long term care facility, hospital, home, or special unit caring for spinal cord injuries, or head injuries. It can be used with patients who are in a comatose condition whereby hip flexion, weight bearing, or transferring to a bedside commode is impossible. This Bag will capture the waste for the lab for testing and other lab work and it is marked “Biohazard Waste Material”. The Bag fits any age of patient, male or female.

### BACKGROUND OF THE INVENTION

1. Field of the Invention

The present invention relates to a human body fluids waste bag.

The use of the Enema Body Fluid (E.B.F.) Bag will allow the caregiver to administer the enema and collect contents without contamination of patient and/or caregiver. The E.B.F. Bag will collect all fecal materials and will measure for accurate records. The E.B.F. Bag will allow easy clean-up following enema and disposal of contents without risk of contamination to patient and health care provider. The E.B.F. Bag will prevent the body fluids/waste from contaminating bed linens and/or the patient. The E.B.F. Bag identifies this as a Biohazard Waste Bag. The E.B.F. Bag comes in different sizes. The E.B.F. Bag will capture the waste for disposal. The E.B.F. Bag will decrease cost of maintaining healthy skin by preventing secondary skin breakdown due to contact of fecal matter with patient's skin.

### SUMMARY OF THE INVENTION

Because of the disadvantages inherent in the current types of human enema waste collecting bag the E.B.F. Bag was created. The general purpose of the present invention, which will be described subsequently in greater detail, is to provide a new and improved way to collect human waste from many patients even in difficult situations. These situations include, but are not limited to, those patients unable to ambulate, sit upright or transfer to a bedside commode.

Obtaining this patent for the present invention essentially benefits a patient with a medical condition and the health professional administering the enema procedure.

The above outlines rather broadly the major features of the invention in order that the invention's contribution to medical science may be better appreciated. There are, of course, additional features of the invention that will be described hereinafter and which will form the subject matter of the claims appended hereto.

In this respect, before explaining the invention in detail, it is to be understood that the invention is not limited in its application to the details of construction or to the arrangements of the components set forth in the following description or illustrated in the drawings. The invention is capable of other applications and of being utilized in various ways. Also, it is to be understood that the phraseology and terminology employed herein are for the purpose of description and should not be regarded as limiting. It is important that the claims be regarded as including equivalent constructions.

Further, the purpose of the foregoing abstract is to enable the U.S. Patent and Trademark Office and the public generally, and especially the scientists, engineers and practitioners in the art who are not familiar with patent or legal terms or phraseology, to determine quickly from a cursory inspection the nature and essence of the technical disclosure of the application. The abstract is neither intended to define the invention of the application, which is measured by the claims, not is it intended to be limiting as to the scope of the invention in any way.

It is therefore an object of the present invention to provide a new procedure to administer the enema and collect the contents without contamination to the patient and/or the caregiver and none of the disadvantages.

It is another object of the present invention to provide a new way to control medical costs.

It is a further object of the present invention to provide for more durable and reliable construction.

Further object of the present invention is to provide a new procedure which eliminates expensive surgery and additional medical expenses.

Still another object of the present invention is to provide an affordable alternative to surgery for all hospitals, nursing homes, out-patient and other facilities requiring this procedure. It is also cost effective for the medical facility itself by reducing overhead costs.

These, together with other objects of the invention, along with the various features of novelty which characterize the invention, are pointed out with particularity in the claims annexed to and forming a part of this disclosure. For a better understanding of the invention, its operating advantages and the specific objects attained by its uses, reference should be had to the accompanying drawings and descriptive matter in which there is illustrated preferred embodiments of the invention.

### BRIEF DESCRIPTION OF THE DRAWINGS

The invention will be better understood and objects other than those set forth above will become apparent when consideration is given to the following detailed description thereof. Such description makes reference to the annexed drawings wherein:

FIG. 1 is a perspective view of a first embodiment of enema body fluid bag comprising the present invention.

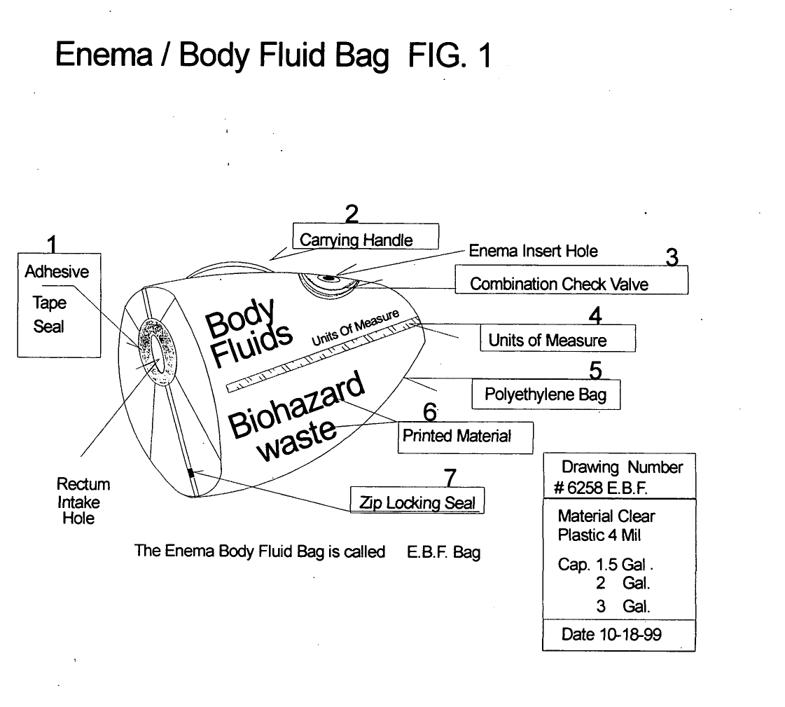

FIG. 2 is a cross-sectional view of the invention viewed as a cut-away view of the plastic enema bag of FIG. 1.

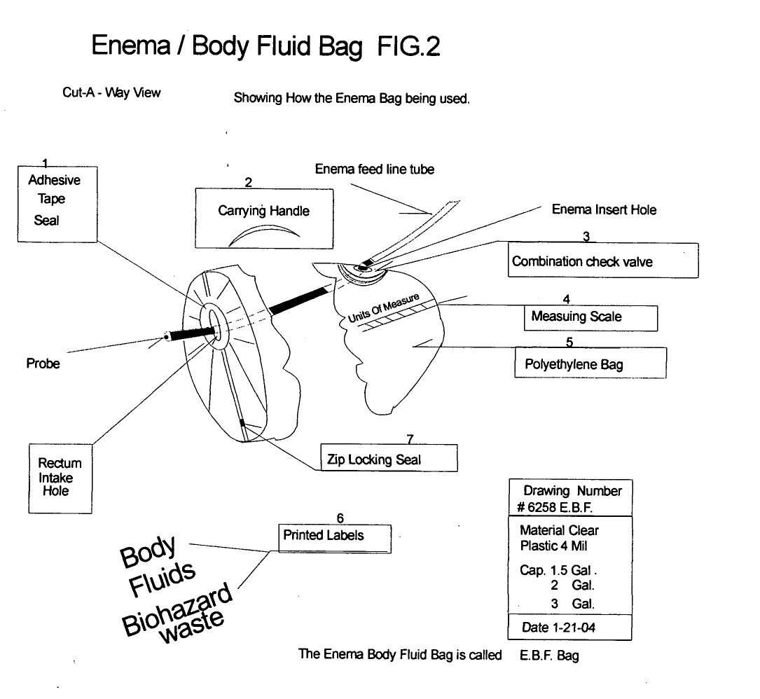

1. A new human body fluid waste material collecting bag comprising.
a first polyethylene material bag that will allow an enema to be administered through the bag and collect the human body fluid waste.
a second combination check valve that will allow the enema probe to be inserted through the check valve into the bag cavities, then through the adhesive tape seal, then into the human rectum to administer the enema procedure.
a third zip locking seal to secure the waste material from spilling or leaking.

1. The new enema body fluid bag known as E.B.F. Bag that collects human body waste as described in claim 1, wherein said bag includes an adhesive tape seal item #1 that attaches to the human rectum whereas the enema probe will be inserted through the bag into the human rectum.

2. The new enema body fluid bag known as E.B.F. Bag that collects human body waste as described in claim 1, wherein said bag includes a carrying handle for transporting the waste material item #2.

3. The new enema body fluid bag known as E.B.F. Bag that collects human body waste as described in claim 1, wherein said bag includes combination check valve known as the duck bill valve item #3 that allows the enema probe to be inserted into the plastic bag then through the plastic bag through adhesive tape seal that is attached to the human rectum.

4. The new enema body fluid bag known as E.B.F. Bag that collects human body waste as described in claim 1, wherein said bag includes a measuring scale item #4 that will read how much body waste has been collected.

5. The new enema body fluid bag known as the E.B.F. Bag that collects human body waste as described in claim 1, wherein said bag includes the polyethylene bag item #5 which will collect the body waste while the enema is being administered.

6. The new enema body fluid bag known as E.B.F. Bag that collects human body waste as described in claim 1, wherein said bag includes printed material item #6 that indicates the units of measure, and identifies that the bag contains body fluids and biohazard waste.

7. The new enema body fluid bag known as E.B.F. Bag that collects human body waste as described in claim 1, wherein said bag includes a zip locking closure item #7 that will secure the waste material in the bag from spilling or leaking while being transported to lab or while disposing of the body waste fluids.
